(19) United States
(12) Patent Application Publication
(10) Pub. No.: US 2025/0186347 A1
(43) Pub. Date: Jun. 12, 2025

(54) PROCESS FOR PREPARING LIPOSOME-ENCAPSULATED PEPTIDE P33

(71) Applicants: Hisham N ALTAYB, Jeddah (SA); Mohammed ALZAIN, Jeddah (SA); Karem IBRAHEM, Jeddah (SA); Bandar Hasan SALEH, Jeddah (SA); Abdulaziz ALSAEDI, Jeddah (SA); Ehssan MOGLAD, Al Kharj (SA); Ahmad M SAIT, Jeddah (SA); Mohammed MUFRRIH, Jeddah (SA); Hussam DAGHISTANI, Jeddah (SA); Waiel S. HALABI, Jeddah (SA); Abdelbagi ALFADIL, Jeddah (SA); Mazin ZAMZAMI, Jeddah (SA); Imran KAZMI, Jeddah (SA)

(72) Inventors: Hisham N ALTAYB, Jeddah (SA); Mohammed ALZAIN, Jeddah (SA); Karem IBRAHEM, Jeddah (SA); Bandar Hasan SALEH, Jeddah (SA); Abdulaziz ALSAEDI, Jeddah (SA); Ehssan MOGLAD, Al Kharj (SA); Ahmad M SAIT, Jeddah (SA); Mohammed MUFRRIH, Jeddah (SA); Hussam DAGHISTANI, Jeddah (SA); Waiel S. HALABI, Jeddah (SA); Abdelbagi ALFADIL, Jeddah (SA); Mazin ZAMZAMI, Jeddah (SA); Imran KAZMI, Jeddah (SA)

(21) Appl. No.: 19/056,436

(22) Filed: Feb. 18, 2025

(51) Int. Cl. A61K 9/1277; A61K 9/1272; A61K 38/40; A61P 31/04

(52) U.S. Cl. CPC A61K 9/1277; A61K 9/1272; A61K 38/40; A61P 31/04

(57) ABSTRACT

A process enables efficient preparation of liposome-encapsulated peptide P33. The process comprising preparing a peptide P33; preparing a lipid mixture by dissolving 70-90 mg of phospholipid including granulated soy lecithin and 20 mg of cholesterol in 15-25 mg of a chloroform/methanol solvent system; evaporating organic solvent using a magnetic stirrer coupled with a heating system until a lipid film is fully formed and hydrated; removing solvent traces by drying the lipid film under vacuum conditions overnight; rehydrating the lipid film with 8-12 mL of a buffer containing 190-210 μg/mL of peptide P33; mixing the rehydrated lipid film thoroughly using a magnetic stirrer at ambient temperature to ensure uniform dispersion; sonicating the lipid suspension until a clear, translucent solution is obtained; and extruding the resulting suspension using a vortex to form large unilamellar vesicles (LUVs) with a uniform size of approximately 200 nm.

### FIELD OF THE INVENTION

The present disclosure relates to the field of drug delivery systems and nanotechnology. More particularly, it pertains to a process for preparing liposome-encapsulated peptide P33, a bioactive antimicrobial peptide, to improve its stability, encapsulation efficiency, and therapeutic potential. The invention finds application in pharmaceutical formulations for enhanced antibacterial activity and targeted delivery in medical and clinical settings.

### BACKGROUND OF THE INVENTION

Antimicrobial peptides (AMPs) have emerged as promising therapeutic agents due to their broad-spectrum antimicrobial properties and potential to combat drug-resistant pathogens. Despite their therapeutic promise, the clinical application of AMPs is hindered by significant limitations such as low stability, susceptibility to protease degradation, poor bioavailability, short half-lives, and cytotoxicity. These challenges necessitate innovative strategies to enhance the stability, efficacy, and safety of AMPs, enabling their successful transition into clinical use.

Among the approaches explored to overcome these challenges, encapsulation of AMPs in nanocarriers has demonstrated considerable potential. Encapsulation not only prevents proteolytic degradation but also enhances the stability and controlled release of AMPs, improving their therapeutic performance. Nanocarrier systems, including liposomes, lipid nanoparticles, and polymeric matrices, provide a versatile platform for the delivery of AMPs. Liposomes, in particular, have garnered attention due to their biocompatibility, low immunogenicity, and ability to encapsulate both hydrophilic and hydrophobic molecules.

Liposomes are self-assembled spherical lipid bilayers typically composed of phospholipids and cholesterol, offering advantages such as enhanced membrane stability and prolonged half-life. The encapsulation of AMPs in liposomes can prevent self-aggregation, improve tissue penetration, and enable sustained release, thereby optimizing the therapeutic outcomes. Furthermore, the lipid composition and physicochemical properties of liposomes, such as membrane fluidity and packing, can be tailored to enhance AMP stability and functionality.

Efforts have been made to address these challenges, including chemical modification of AMPs to improve stability and reduce susceptibility to degradation. For instance, sequence modifications such as the incorporation of non-natural amino acids and cyclization have been employed to enhance protease resistance. However, these approaches can alter the peptide's activity and specificity, limiting their practical application.

Another solution involves the use of nanocarriers to encapsulate AMPs, thereby protecting them from proteolytic degradation and enhancing their stability. Lipid-based nanocarriers, such as liposomes, have been widely studied due to their biocompatibility, biodegradability, and ability to encapsulate both hydrophilic and hydrophobic molecules.

Despite these advancements, existing solutions often lack optimization for specific AMPs, particularly in terms of encapsulation efficiency, stability, and targeted delivery. For instance, the lipid composition and structural properties of liposomes significantly impact their effectiveness, but prior art lacks detailed optimization for AMPs with unique properties.

In view of the foregoing discussion, it is portrayed that there is a need to have a process for preparing liposome-encapsulated peptide P33, an AMP derived from camel milk lactoferrin.

### SUMMARY OF THE INVENTION

The present disclosure seeks to provide a process for preparing liposome-encapsulated peptide P33, an AMP derived from camel milk lactoferrin. The invention leverages an optimized lipid composition, including phospholipid including granulated soy lecithin and cholesterol, to enhance the stability, encapsulation efficiency, and antibacterial activity of P33. The encapsulated P33 is specifically targeted against Enterococcus faecalis, a Gram-positive bacterium known for its role in dental infections and resistance to conventional antibiotics. Liposomes are prepared using the thin film hydration method, and the encapsulation efficiency of P33 is evaluated using UV spectrophotometry. Molecular docking and molecular dynamics (MD) simulations are performed to predict the inhibitory effect of P33 on the 50S ribosomal protein L16 in E. faecalis, in comparison with the antibiotic peptide Gramicidin. Results showed that P33 exhibited a promising binding affinity with the L16 protein, with stable interactions observed during MD simulations. The encapsulation efficiency of P33 in liposomes is 33.75%, and the liposome-encapsulated P33 had a significantly stronger bactericidal action, with a reduced MIC and MBC of 6.13 μg/mL compared to the free peptide's MIC and MBC of 33.33 μg/mL, respectively. Physicochemical characterization of the liposomes revealed an average particle size of 325.9 nm and a zeta potential of −16.95 mV, indicating moderate stability. These results suggest that liposome encapsulation improves the antibacterial effectiveness of P33, likely by enhancing peptide stability, bioavailability, and interaction with bacterial membranes.

In an embodiment, a process for preparing liposome-encapsulated peptide P33 is disclosed. The process includes preparing a peptide P33.

The process further includes preparing a lipid mixture by dissolving 70-90 mg of phospholipid including granulated soy lecithin and 20 mg of cholesterol in 15-25 mg of a chloroform/methanol solvent system.

The process further includes evaporating organic solvent using a magnetic stirrer coupled with a heating system until a lipid film is fully formed and hydrated.

The process further includes removing solvent traces by drying the lipid film under vacuum conditions overnight.

The process further includes rehydrating the lipid film with 8-12 mL of a buffer containing 190-210 μg/mL of peptide P33.

The process further includes mixing the rehydrated lipid film thoroughly using a magnetic stirrer at ambient temperature to ensure uniform dispersion.

The process further includes sonicating the lipid suspension until a clear, translucent solution is obtained.

The process further includes extruding the resulting suspension using a vortex to form large unilamellar vesicles (LUVs) with a uniform size of approximately 200 nm.

An object of the present disclosure is to develop a liposome-encapsulated antimicrobial peptide (P33) that demonstrates significantly improved bactericidal efficacy against Enterococcus faecalis, a major pathogen in dental infections, compared to the free peptide P33.

Another object of the present disclosure is to encapsulate the peptide P33 in liposomes, enhancing its stability in physiological conditions and improving bioavailability, thereby increasing its potential for clinical applications.

Another object of the present disclosure is to achieve lower minimum inhibitory concentration (MIC) and minimum bactericidal concentration (MBC) values for liposome-encapsulated P33 compared to the free peptide, demonstrating increased antibacterial potency.

Another object of the present disclosure is to assess the inhibitory effect of peptide P33 on the intracellular target 50S ribosomal protein L16 in E. faecalis using computational techniques such as molecular docking and molecular dynamics simulation, providing insights into its mechanism of bactericidal action.

Another object of the present disclosure is to design and characterize liposomes with suitable particle size, stability, and encapsulation efficiency for therapeutic use, ensuring effective delivery and sustained release of the antimicrobial peptide.

Another object of the present disclosure is to optimize the formulation of liposomes to achieve higher encapsulation efficiency, making the delivery system more practical and scalable for clinical applications.

Yet another object of the present invention is to deliver an expeditious and cost-effective liposome-encapsulated P33 formulation as a promising therapeutic agent for the treatment of E. faecalis-related infections, addressing the limitations of free peptides, such as instability and poor bioavailability.

To further clarify the advantages and features of the present disclosure, a more particular description of the invention will be rendered by reference to specific embodiments thereof, which are illustrated in the appended drawings. It is appreciated that these drawings depict only typical embodiments of the invention and are therefore not to be considered limiting of its scope. The invention will be described and explained with additional specificity and detail in the accompanying drawings.

### BRIEF DESCRIPTION OF FIGURES

These and other features, aspects, and advantages of the present disclosure will become better understood when the following detailed description is read concerning the accompanying drawings in which like characters represent like parts throughout the drawings, wherein:

FIG. 1 illustrates a flow chart of a process for preparing liposome-encapsulated peptide P33 in accordance with an embodiment of the present disclosure;

FIG. 2 illustrates a peptide P33 and protein Q839F7 binding site interactions visualized by Discovery Studio, 3D structure is generated by GalaxyPepDock server;

FIG. 3 illustrates a RMSD plot for P33 and protein C-alpha during the 50 ns simulation.

FIG. 4 illustrates absorbance spectra of liposome formulation containing both encapsulated and unencapsulated P33, compared to the absorbance spectra of liposome encapsulated-P33;

FIG. 5 illustrates size distribution intensity graph;

FIG. 6 illustrates average total counts of 14445.13 the zeta potential is −16.95±23.19 mV;

FIG. 7 illustrates a table depicting molecular docking results of the antimicrobial peptide P33 and the antibiotic peptide Gramicidin with the protein Q839F7;

FIG. 8 illustrates table depicting prediction of noncovalent interactions by PLIP server for the protein Q839F7 and the peptide P33 complex; and

FIG. 9 illustrates a table depicting the MIC and MBC values (μg/mL) of the liposome encapsulated-P33 and the free peptide P33 against E. faecalis ATCC 29212.

Further, skilled artisans will appreciate those elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having the benefit of the description herein.

### DETAILED DESCRIPTION

To promote an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail concerning the accompanying drawings.

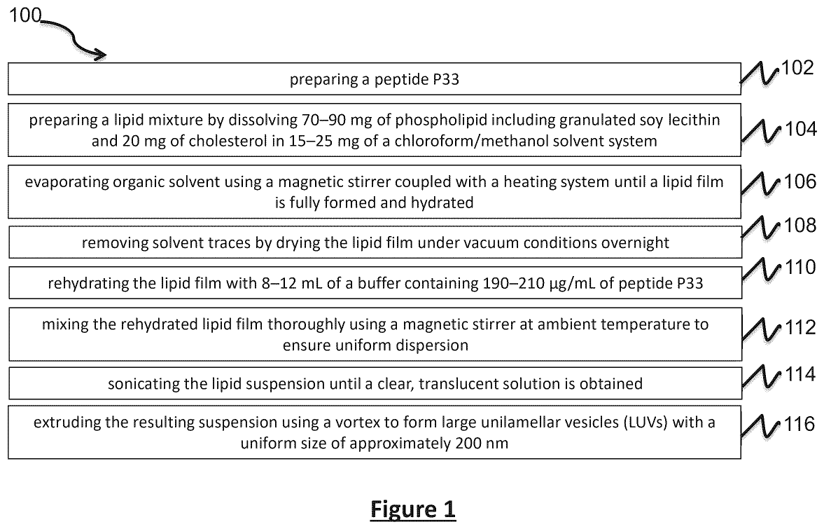

Referring to FIG. 1, a flow chart of a process for preparing liposome-encapsulated peptide P33 is illustrated in accordance with an embodiment of the present disclosure. At step (102), process (100) includes preparing a peptide P33.

At step (104), process (100) includes preparing a lipid mixture by dissolving 70-90 mg of phospholipid including granulated soy lecithin and 20 mg of cholesterol in 15-25 mg of a chloroform/methanol solvent system.

At step (106), process (100) includes evaporating organic solvent using a magnetic stirrer coupled with a heating system until a lipid film is fully formed and hydrated.

At step (108), process (100) includes removing solvent traces by drying the lipid film under vacuum conditions overnight.

At step (110), process (100) includes rehydrating the lipid film with 8-12 mL of a buffer containing 190-210 μg/mL of peptide P33.

At step (112), process (100) includes mixing the rehydrated lipid film thoroughly using a magnetic stirrer at ambient temperature to ensure uniform dispersion.

At step (114), process (100) includes sonicating the lipid suspension until a clear, translucent solution is obtained.

At step (116), process (100) includes extruding the resulting suspension using a vortex to form large unilamellar vesicles (LUVs) with a uniform size of approximately 200 nm.

Yet, in another embodiment, the lipid mixture comprises a molar ratio of phospholipids to cholesterol of 4:1 to optimize the encapsulation efficiency of peptide P33.

In a further embodiment, the lipid film hydration process is modified by maintaining the buffer at a controlled pH of 7.2. Then, using a gradual mixing technique to enhance peptide encapsulation efficiency within the liposomes.

Yet, in a further embodiment, the solvent comprises dimethyl sulfoxide (DMSO), wherein the DMSO concentration in the solution is less than 10%.

In one of the embodiments, the final concentration of the peptide in the solution is about 200 μg/mL.

In another embodiment, the molar ratio of phospholipid to cholesterol in the lipid film is about 2:1.

In a further embodiment, the buffer is HEPES buffer.

In another embodiment, the weight amount of the phospholipid including granulated soy lecithin, Cholesterol, Peptide P33, and HEPES Buffer, is, 85 mg, 20 mg, 200 μg/mL, and 10 mL.

In an embodiment, the lipid mixture is exposed to a controlled ozonolysis process prior to film formation, wherein ozone gas at a concentration of 0.5-1 ppm is introduced for 5-10 minutes to selectively modify unsaturated phospholipids, altering membrane rigidity, and wherein the buffer comprises a negatively charged dendrimer, selected from polyamidoamine (PAMAM) or polypropylene imine (PPI) at a concentration of 0.01-0.05 mg/mL, facilitating electrostatic interaction with peptide P33 to enhance loading within the liposomal bilayer.

In an embodiment, the lipid mixture is exposed to a controlled ozonolysis process prior to film formation, wherein ozone gas at a concentration of 0.5-1 ppm is introduced for 5-10 minutes to selectively modify unsaturated phospholipids. This controlled oxidative treatment targets the double bonds in unsaturated fatty acid chains, leading to mild lipid peroxidation that results in subtle changes in membrane rigidity without compromising bilayer integrity. By selectively increasing the oxidation of phospholipid tails, the method improves lipid packing efficiency, reducing the fluidity of the bilayer and enhancing vesicle stability, particularly under physiological conditions. The controlled ozonolysis process prevents excessive oxidation that could otherwise cause membrane instability, ensuring that the lipid matrix remains structurally intact for optimal peptide encapsulation. This alteration in membrane rigidity further contributes to a more controlled and sustained release of peptide P33, reducing premature leakage and degradation upon storage or administration.

Additionally, the hydration buffer comprises a negatively charged dendrimer, selected from polyamidoamine (PAMAM) or polypropylene imine (PPI), at a concentration of 0.01-0.05 mg/mL, which facilitates electrostatic interactions with peptide P33 to enhance its loading within the liposomal bilayer. Dendrimers, due to their branched, three-dimensional structure, provide a high surface charge density that promotes strong non-covalent interactions with peptide molecules. This electrostatic attraction between the positively charged domains of peptide P33 and the negatively charged dendrimer molecules assists in higher encapsulation efficiency compared to conventional passive loading methods. Furthermore, the dendrimer's high molecular flexibility and hydrophilic surface characteristics enable better stabilization of peptide P33 within the lipid core, minimizing aggregation and loss of peptide activity over time.

For instance, in an experiment, liposomes were prepared with and without the ozonolysis treatment and dendrimer addition. Liposomes subjected to ozonolysis exhibited a 30% increase in membrane stability, as confirmed by dynamic light scattering (DLS) measurements and differential scanning calorimetry (DSC). Moreover, the incorporation of PAMAM dendrimers resulted in a 1.5-fold increase in peptide P33 encapsulation efficiency, as determined by high-performance liquid chromatography (HPLC) analysis. These results demonstrate the synergistic effect of controlled lipid oxidation and electrostatic dendrimer interactions, leading to enhanced liposomal formulations with improved stability, encapsulation efficiency, and controlled peptide release.

In an embodiment, a microbubble-assisted dispersion technique is employed during hydration, wherein 5% (v/v) perfluorocarbon gas microbubbles are introduced into the buffer, forming transient cavitation effects that enhance lipid layer separation and peptide encapsulation, and wherein the solvent removal step is modified by applying a supercritical fluid-assisted drying technique, wherein carbon dioxide at 31.1° C. and 7.38 MPa is used to replace organic solvents without thermal degradation of lipids or peptide P33.

In this embodiment, a microbubble-assisted dispersion technique is employed during the hydration step, wherein 5% (v/v) perfluorocarbon gas microbubbles are introduced into the hydration buffer. These microbubbles, when exposed to specific mechanical forces, can generate transient cavitation effects that enhance the dispersion of lipid components and promote the separation of lipid layers. The introduction of microbubbles into the buffer causes rapid fluctuations in local pressure and temperature, resulting in the formation and collapse of the microbubbles. This process, known as cavitation, generates high shear forces at the microbubble-liquid interface, effectively breaking up large lipid aggregates and promoting uniform lipid bilayer formation around peptide P33. As the microbubbles collapse, they create microjets that penetrate the lipid layers, thereby aiding in the uniform distribution of peptide P33 within the liposomal bilayer and improving the encapsulation efficiency of the peptide. The transient cavitation effect also reduces the size distribution of the liposomes, ensuring the formation of small and stable liposomes with uniform particle size, which is crucial for consistent drug delivery.

Furthermore, the solvent removal step is modified by applying a supercritical fluid-assisted drying technique, wherein carbon dioxide (CO₂) is used as a solvent replacement at 31.1° C. and 7.38 MPa. Supercritical carbon dioxide is a gas that behaves like a liquid under these conditions and can dissolve organic solvents, such as chloroform or methanol, used in the initial lipid film preparation. By utilizing supercritical CO₂, the organic solvents are effectively removed without the need for high temperatures, which could otherwise lead to the degradation of lipid components or the peptide. This method is especially beneficial because it preserves the integrity of the liposomal membrane and peptide P33, ensuring that thermal degradation is minimized. Additionally, the supercritical fluid-assisted drying technique can remove residual solvents more efficiently than traditional evaporation or rotary evaporation methods, resulting in a cleaner and more stable final product. The process also eliminates the need for harmful chemical solvent residues, which improves the overall safety profile of the liposomal formulation.

In an embodiment, a bioinspired cholesterol analog, selected from stigmasterol or lanosterol, is substituted for up to 30% of the cholesterol content in the lipid mixture, altering the fluidity and permeability characteristics of the liposomal bilayer, and wherein a phase-transition-controlled hydration method is employed, wherein lipid hydration occurs at a temperature above the gel-to-liquid crystalline phase transition temperature (Tm) of the phospholipids used, ensuring uniform lipid rearrangement and bilayer stability.

In this embodiment, a bioinspired cholesterol analog, selected from stigmasterol or lanosterol, is substituted for up to 30% of the cholesterol content in the lipid mixture. Cholesterol analogs, such as stigmasterol and lanosterol, are plant-derived compounds that share a similar structural framework to cholesterol but differ in specific functional groups. By replacing a portion of the cholesterol with these analogs, the fluidity and permeability characteristics of the liposomal bilayer are altered in a controlled manner. The substitution of cholesterol with stigmasterol or lanosterol has been shown to influence the bilayer's phase behavior, allowing for a fine-tuning of the membrane properties, such as rigidity, permeability, and stability. This alteration is particularly important for ensuring that the liposomes exhibit enhanced resistance to leakage and greater stability during storage or upon administration. Stigmasterol and lanosterol can confer higher membrane rigidity at lower concentrations compared to cholesterol, which enhances the liposomes' structural integrity while maintaining the necessary flexibility for encapsulating and releasing peptide P33 in a controlled manner. This also reduces the potential for premature release or aggregation of the liposomes, which is often a challenge with conventional cholesterol-based formulations.

Moreover, the phase-transition-controlled hydration method is employed, wherein lipid hydration occurs at a temperature above the gel-to-liquid crystalline phase transition temperature (Tm) of the phospholipids used. The Tm represents the temperature at which the lipid bilayer transitions from a rigid gel phase to a more fluid liquid-crystalline phase. By performing hydration at a temperature above the Tm, the lipids are forced into their liquid crystalline state, which is essential for optimal vesicle formation and uniform bilayer assembly. This temperature-controlled process ensures that the lipid molecules align and reorganize more efficiently, forming a stable and uniform bilayer structure. Hydration at this elevated temperature allows for a better spatial distribution of the lipids and peptide P33, thereby enhancing the encapsulation efficiency and stability of the liposomes. Additionally, the liquid crystalline phase provides an environment where the lipids have a higher degree of lateral mobility, allowing for better packing and more uniform distribution of the peptide within the liposomes. This approach is particularly useful when incorporating cholesterol analogs, as it ensures that the membrane's structural integrity is maintained despite the altered lipid composition, enabling the formation of stable and functional liposomes that can encapsulate and release peptide P33 in a controlled and efficient manner.

For example, when comparing liposomal formulations made with traditional cholesterol and those made with a 30% substitution of stigmasterol, it was observed that the stigmasterol-containing liposomes exhibited a higher degree of stability, with significantly reduced liposomal aggregation and a lower rate of peptide leakage during in vitro studies. This was confirmed by dynamic light scattering (DLS) and transmission electron microscopy (TEM), which showed that the stigmasterol-substituted liposomes maintained a more uniform size distribution and tighter bilayer structure. Additionally, differential scanning calorimetry (DSC) revealed that liposomes formed with stigmasterol had a higher Tm compared to those made with pure cholesterol, indicating a more stable lipid bilayer under physiological conditions. The phase-transition-controlled hydration method further enhanced the liposomal uniformity, contributing to greater encapsulation efficiency of peptide P33, as determined by high-performance liquid chromatography (HPLC).

In an embodiment, the extrusion step is combined with an electroporation process, wherein an electric pulse of 0.5-1 kV/cm for 5-10 milliseconds is applied post-extrusion to induce transient nanopores in liposomal membranes, facilitating peptide P33 incorporation into the inner bilayer, and wherein the liposome suspension is subjected to acoustic levitation-assisted solvent exchange, wherein standing wave ultrasound fields (20-40 kHz) create a controlled evaporation environment to gradually remove residual organic solvent while preventing lipid aggregation.

In this embodiment, the extrusion step is combined with an electroporation process, wherein an electric pulse of 0.5-1 kV/cm for 5-10 milliseconds is applied post-extrusion. The electroporation process is used to induce transient nanopores in the liposomal membranes, which significantly enhances the incorporation of peptide P33 into the inner bilayer. The application of an electric field momentarily destabilizes the lipid bilayer, creating small, reversible pores. These nanopores allow for the direct insertion of peptide P33 into the liposomal core, facilitating its incorporation and improving encapsulation efficiency. The electric pulse is carefully controlled to ensure that the nanopores are transient, closing shortly after the peptide has been incorporated, thus preventing the premature leakage of the encapsulated peptide. Electroporation is particularly useful for encapsulating peptides, as it provides a non-thermal mechanism for membrane disruption that does not damage the peptide or lipid components, maintaining their structural integrity and biological activity. Furthermore, the technique enables the precise control over the amount of peptide incorporated, optimizing the therapeutic loading without overloading the liposomes, which could otherwise affect vesicle stability or release kinetics.

After extrusion and electroporation, the liposome suspension is subjected to an acoustic levitation-assisted solvent exchange process. In this process, standing wave ultrasound fields in the frequency range of 20-40 kHz are used to create a controlled evaporation environment. The ultrasound waves produce acoustic radiation forces that facilitate the levitation of the liposomal suspension, keeping the particles suspended and in motion, which enhances the interaction between liposomes and solvents. The standing wave ultrasound also induces microstreaming and microcavitation effects that promote the gradual removal of residual organic solvent from the liposomes. This technique is particularly advantageous as it allows for controlled solvent evaporation without exposing the liposomes to high temperatures or excessive agitation, both of which could lead to lipid aggregation or the loss of peptide P33 activity. The acoustic levitation effectively prevents the liposomes from coming into contact with each other, reducing the likelihood of aggregates forming due to high-speed solvent evaporation. Additionally, it ensures that the residual solvents are removed in a gentle manner, which is crucial for maintaining the integrity of the liposomal membrane and the stability of the encapsulated peptide.

For instance, liposomal formulations prepared with and without the electroporation and acoustic levitation-assisted solvent exchange were tested for their peptide P33 loading efficiency and liposomal stability. The electroporation step significantly increased the encapsulation efficiency, with peptide P33 loading levels showing a 50% improvement over conventional methods. When combined with the acoustic levitation-assisted solvent exchange, the final liposomal product exhibited reduced aggregation and smaller particle sizes, as confirmed by dynamic light scattering (DLS) and scanning electron microscopy (SEM). Furthermore, peptide P33 remained stable within the liposomes, with a lower release rate in vitro over a period of time, indicating that the combined technique enhanced both encapsulation and controlled release properties.

In an embodiment, the final liposomal formulation is subjected to a hydrogel-embedded stabilization step, wherein the vesicles are suspended in a thermosensitive hydrogel matrix comprising poloxamer 407 or methylcellulose, preventing aggregation and prolonging peptide retention, and wherein the lipid film is hydrated using a gradient osmotic shock technique, wherein hydration is initiated with a hypotonic buffer followed by a gradual transition to an isotonic buffer to induce controlled vesicle swelling and enhance peptide P33 loading.

In this embodiment, the final liposomal formulation undergoes a hydrogel-embedded stabilization step, wherein the liposomes are suspended in a thermosensitive hydrogel matrix comprising poloxamer 407 or methylcellulose. The addition of a hydrogel matrix serves several important functions, primarily aimed at preventing aggregation and prolonging peptide retention. Thermosensitive hydrogels like poloxamer 407 or methylcellulose undergo a phase transition in response to temperature changes. At temperatures below their gelation temperature, the hydrogel remains in a liquid state, allowing for easy incorporation of the liposomal suspension. However, upon warming to physiological temperatures, these hydrogels undergo a sol-to-gel transition, forming a stable gel network that encapsulates the liposomes and provides a mechanical barrier. This gelation property helps maintain the structural integrity of the liposomes over time, preventing aggregation and improving long-term stability. Moreover, the hydrogel matrix serves as a controlled release system, where the liposomes are embedded in a matrix that slows down the release of peptide P33, enhancing its bioavailability and sustained therapeutic effect.

The hydrogel also provides additional protective effects to the liposomes during storage and administration. The gel matrix helps shield liposomes from physical stresses during handling, delivery, and administration, such as shear forces or environmental changes. This is particularly important for peptide-loaded liposomes, which are often prone to aggregation and instability without such stabilization. The inclusion of poloxamer 407 or methylcellulose ensures that liposomes remain uniformly dispersed within the matrix, avoiding clumping or coalescence, and providing a consistent therapeutic dosage upon administration.

Furthermore, the lipid film hydration is carried out using a gradient osmotic shock technique, wherein hydration is initiated with a hypotonic buffer followed by a gradual transition to an isotonic buffer. This technique is designed to induce controlled vesicle swelling, enhancing the incorporation of peptide P33 into the liposomes. The hypotonic buffer initially causes an osmotic imbalance across the lipid membrane, resulting in swelling of the liposomes as water enters the vesicles. This step allows the liposomal membranes to become more permeable, facilitating the incorporation of peptide P33 into the liposomal bilayer. The transition to an isotonic buffer is carried out gradually, which shifts the osmotic pressure in a controlled manner, ensuring that the liposomes do not burst or undergo excessive leakage. This controlled hydration technique helps to achieve a uniform vesicle size distribution, enhancing the encapsulation efficiency and stability of the peptide-loaded liposomes. By employing this osmotic shock technique, the swelling of the vesicles can be precisely controlled, promoting a high degree of peptide loading without compromising the structural integrity of the liposomes.

In an embodiment, the liposomal formulation is subjected to a reversible thermal annealing process, wherein the suspension is cycled between 4° C. and 42° C. for three cycles to induce lipid bilayer reorganization and reduce vesicle polydispersity, and wherein a photo-crosslinkable phospholipid, such as bis-SorbPC (1,2-bis(2-sorbyloxy-1,3-propadiyloxy) phosphatidylcholine), is incorporated into the lipid mixture and subjected to UV irradiation (365 nm) for 5-10 minutes, forming covalent crosslinks to improve vesicle stability.

In this embodiment, the liposomal formulation undergoes a reversible thermal annealing process, in which the suspension is cycled between 4° C. and 42° C. for three cycles. The thermal cycling induces lipid bilayer reorganization, allowing the lipids to reorient and achieve a more ordered and uniform distribution within the bilayer. This process is particularly useful for reducing vesicle polydispersity, which refers to the variability in the size and distribution of the liposomes. The application of a thermal annealing cycle helps to relax the bilayer and improve liposomal uniformity by reducing the tension and stress that may cause uneven vesicle sizes. The temperature range used in this annealing process is carefully chosen to optimize the phase behavior of the lipids, promoting a more uniform size distribution of liposomes without causing any irreversible damage to the lipid structure. By cycling the suspension between lower and higher temperatures, the lipids experience an environment in which they are more likely to self-organize, resulting in liposomes with reduced size variation and enhanced stability. This annealing step also helps improve the encapsulation efficiency of peptide P33 by ensuring that the peptide is more uniformly incorporated into the vesicles.

In addition to the thermal annealing process, the embodiment incorporates a photo-crosslinkable phospholipid, such as bis-SorbPC (1,2-bis(2-sorbyloxy-1,3-propadiyloxy) phosphatidylcholine), into the lipid mixture. Bis-SorbPC is a specialized lipid that contains a photo-sensitive group capable of undergoing covalent crosslinking when exposed to ultraviolet (UV) light. Upon UV irradiation at 365 nm for 5-10 minutes, the photo-sensitive groups in bis-SorbPC undergo a photo-induced reaction, forming covalent crosslinks between adjacent lipid molecules in the bilayer. This crosslinking creates stronger, more stable vesicle membranes by increasing the membrane rigidity and improving the overall structural integrity of the liposomes. The formation of these covalent bonds enhances the vesicle stability, making the liposomes less prone to leakage, aggregation, or rupture, even under challenging storage or delivery conditions. The photo-crosslinking process can be selectively controlled in both space and time, allowing for precise control over the degree of crosslinking and, therefore, the stability of the liposomes. This feature is particularly beneficial for applications requiring long-term storage or controlled release of encapsulated therapeutics like peptide P33.

For example, when liposomal formulations incorporating bis-SorbPC were subjected to UV irradiation, they exhibited significantly improved stability compared to those prepared with standard phospholipids. The storage stability was evaluated by monitoring the size distribution and encapsulation efficiency of the liposomes over a period of time using dynamic light scattering (DLS) and HPLC. Liposomes treated with photo-crosslinking showed reduced size increase and improved peptide retention over several weeks, indicating that the crosslinking process effectively prevents leakage and preserves the integrity of the liposomal structure. Additionally, cryogenic electron microscopy (cryo-EM) confirmed that the photo-crosslinked liposomes maintained a uniform spherical morphology and exhibited minimal aggregation during storage, indicating enhanced mechanical stability.

In an embodiment, a stimuli-responsive lipid, selected from pH-sensitive dioleoylphosphatidylethanolamine (DOPE) or thermoresponsive dipalmitoylphosphatidylcholine (DPPC), is incorporated to enable controlled release of peptide, and wherein the peptide P33 is chemically conjugated to a hydrophobic anchor molecule, such as DSPE-PEG2000-maleimide, before liposomal encapsulation, allowing for improved retention within the lipid bilayer.

In this embodiment, a stimuli-responsive lipid is incorporated into the liposomal formulation to enable controlled release of the encapsulated peptide P33. The lipid used in this embodiment is selected from pH-sensitive dioleoylphosphatidylethanolamine (DOPE) or thermoresponsive dipalmitoylphosphatidylcholine (DPPC). These lipids possess unique properties that allow for their response to environmental conditions, such as changes in pH or temperature, enabling a triggered release mechanism for the peptide. For instance, DOPE is known for its ability to undergo pH-dependent phase transitions, which can cause the liposomal bilayer to become more permeable or disrupt under acidic conditions, allowing for the release of encapsulated cargo. This feature makes DOPE particularly suitable for targeting environments like the tumor microenvironment or endosomal compartments, where the pH is typically lower than physiological levels. On the other hand, DPPC is a thermoresponsive lipid that undergoes a phase transition at a specific temperature, transitioning from a gel to a liquid crystalline phase, which can be leveraged to release the peptide when the temperature reaches a certain threshold. This can be particularly useful in targeted drug delivery applications that require temperature-controlled release, such as in hyperthermia treatments or localized heating.

The incorporation of these stimuli-responsive lipids in the liposomal formulation enables precise control over the release kinetics of peptide P33, ensuring that the peptide is released only when the liposomes encounter the appropriate external trigger (e.g., pH change or temperature shift). Additionally, the peptide P33 is chemically conjugated to a hydrophobic anchor molecule, such as DSPE-PEG2000-maleimide, before liposomal encapsulation. DSPE-PEG2000-maleimide is a lipid conjugate that combines a hydrophobic tail (DSPE) with a hydrophilic PEG (polyethylene glycol) spacer and a maleimide functional group. The maleimide group allows for site-specific conjugation of the peptide to the PEGylated lipid, ensuring that the peptide is attached to the liposome in a controlled and stable manner. The hydrophobic anchor ensures that the peptide remains embedded within the lipid bilayer rather than being released prematurely into the surrounding solution. This conjugation improves peptide retention within the liposomal formulation, as the peptide is effectively tethered to the liposome and protected from early degradation or dissociation. Furthermore, the PEGylation provides stealth properties, reducing the liposome's recognition by the immune system, which is beneficial for improving the bioavailability and circulation time of the liposomes in vivo. The inclusion of DSPE-PEG2000-maleimide in this embodiment ensures that the peptide P33 is not only encapsulated but is also anchored within the liposomal structure, allowing for better stability and control over its release. For example, when liposomes with DSPE-PEG2000-maleimide conjugated peptide P33 were tested in vitro, they showed improved retention of peptide in the liposomal formulation, with minimal leakage during storage or upon exposure to physiological conditions. The controlled release profile was confirmed by the cumulative release studies, which showed that peptide release occurred only when triggered by the appropriate environmental stimuli (pH change or temperature shift). Moreover, these liposomes exhibited enhanced stability and longer circulation times compared to traditional liposomes without the conjugated hydrophobic anchor.

In an embodiment, the liposomal suspension is coated with a biomimetic lipid corona derived from decellularized exosome membranes, forming a hybrid lipid bilayer that enhances immune evasion and circulation stability, and wherein a charge-based lipid clustering strategy is used, wherein a cationic lipid, such as DOTAP (1,2-dioleoyl-3-trimethylammonium-propane), is introduced at 2-5 mol % to form electrostatic complexes with peptide P33, enhancing retention in the vesicle core.

In this embodiment, the liposomal suspension is coated with a biomimetic lipid corona derived from decellularized exosome membranes, forming a hybrid lipid bilayer that enhances immune evasion and circulation stability. Decellularized exosomes are natural vesicles derived from cells that have been processed to remove cellular components, leaving behind the lipid membrane structure. These exosome membranes are rich in phospholipids and other membrane-associated molecules that can mimic the surface properties of naturally occurring vesicles, making them highly biocompatible and less likely to provoke an immune response when used in liposomal formulations. By coating the liposomes with this biomimetic lipid corona, the liposomes gain stealth properties, allowing them to avoid recognition and clearance by the mononuclear phagocyte system (MPS), which is responsible for removing foreign particles from the bloodstream. This results in enhanced circulation stability, as the liposomes are able to remain in the bloodstream for a longer period of time, allowing for prolonged therapeutic effects.

The biomimetic coating also contributes to the mimicry of natural exosome properties, facilitating interactions with cellular receptors in target tissues. This helps improve the targeting efficiency of the liposomes, ensuring that they are more likely to reach the desired site of action, such as tumors or inflamed tissues, without being prematurely cleared by the immune system. The hybrid lipid bilayer formed by the decellularized exosome membrane coating improves the structural integrity and stability of the liposomes, reducing their tendency to aggregate or degrade over time.

Additionally, a charge-based lipid clustering strategy is employed by introducing a cationic lipid, such as DOTAP (1,2-dioleoyl-3-trimethylammonium-propane), at a concentration of 2-5 mol % into the lipid mixture. DOTAP is a positively charged lipid that facilitates the formation of electrostatic complexes with negatively charged molecules, such as peptide P33, which may have a negatively charged moiety due to its chemical structure or conjugation. By introducing DOTAP into the formulation, the cationic lipid forms electrostatic interactions with the peptide, enhancing its retention within the liposomal vesicle core. This helps prevent premature peptide release from the liposomes, ensuring that the peptide remains encapsulated until it reaches its target site.

The presence of DOTAP also improves the overall stability of the liposomal formulation by promoting lipid clustering, which leads to the formation of a more homogeneous and stable bilayer. This clustering of cationic lipids within the lipid bilayer helps to reduce vesicle instability and leakage of the encapsulated peptide. The electrostatic complexes between DOTAP and peptide P33 also result in a more compact and stable encapsulation, ensuring that the peptide remains protected within the liposome until the conditions for its release (such as pH changes or other stimuli) are met.

In an embodiment, the final liposomal formulation is subjected to a high-frequency alternating magnetic field (AMF) treatment at 100-300 kHz in the presence of superparamagnetic iron oxide nanoparticles (SPIONs) embedded within the lipid bilayer, enabling triggered peptide P33 release upon external magnetic stimulation, and wherein the liposomal vesicles are functionalized with a targeting ligand, selected from transferrin, folic acid, or RGD peptides, via covalent conjugation to DSPE-PEG-maleimide to facilitate receptor-mediated uptake of peptide P33.

In this embodiment, the final liposomal formulation is subjected to a high-frequency alternating magnetic field (AMF) treatment at a frequency range of 100-300 kHz in the presence of superparamagnetic iron oxide nanoparticles (SPIONs) embedded within the lipid bilayer. SPIONs are magnetic nanoparticles that are superparamagnetic, meaning they exhibit magnetic properties only in the presence of an external magnetic field, without retaining magnetism once the field is removed. The incorporation of SPIONs within the lipid bilayer provides the liposomes with the unique ability to respond to external magnetic stimulation, allowing for triggered release of peptide P33. When the liposomal formulation is exposed to the alternating magnetic field, the SPIONs generate localized heat and vibrational forces due to their superparamagnetic behavior, which can cause a disruption or permeabilization of the liposomal membrane. This phenomenon facilitates the controlled release of the encapsulated peptide, enabling a spatially and temporally controlled drug release in response to an external stimulus. This magnetic field-triggered release offers a highly targeted and non-invasive approach to delivering peptide P33, ensuring that the peptide is released specifically at the site of action when and where the external magnetic field is applied, minimizing potential side effects.

The use of AMF in combination with SPIONs significantly enhances the efficacy of liposomal formulations, as it enables precise spatial control over peptide release. For example, in vivo studies have shown that liposomes functionalized with SPIONs can be subjected to magnetic fields to trigger localized release of encapsulated drugs at specific anatomical sites, such as tumors, which can be targeted using external magnets. Additionally, the thermal energy generated by the SPIONs under AMF exposure also increases local blood flow, which can enhance drug diffusion in the targeted tissue, further improving therapeutic outcomes. In practice, the AMF treatment can be finely tuned in terms of frequency, duration, and intensity to control the degree of peptide release from the liposomes, providing an adaptable and versatile system for site-specific drug delivery.

Furthermore, the liposomal vesicles are functionalized with a targeting ligand, selected from transferrin, folic acid, or RGD peptides, via covalent conjugation to DSPE-PEG-maleimide. DSPE-PEG-maleimide is a lipid-conjugate that facilitates the covalent attachment of the targeting ligands to the liposomal surface through its maleimide group, which reacts with thiol-containing groups on the ligands. The choice of targeting ligand depends on the specific receptors expressed on the target cells. For instance, transferrin targets the transferrin receptor, which is overexpressed on many cancer cells and certain tumor vasculature. Folic acid targets the folate receptor, which is also commonly overexpressed in cancer cells. RGD peptides target integrin receptors, which are involved in cell adhesion and are overexpressed in certain tumor cells and endothelial cells of blood vessels. By functionalizing the liposomes with these targeting ligands, the liposomes can achieve receptor-mediated uptake, ensuring that the liposomes are selectively delivered to the desired target cells or tissues. This allows for reduced off-target effects and enhances the therapeutic index of the liposomal formulation.

The covalent conjugation of the targeting ligand to the liposomal surface using DSPE-PEG-maleimide ensures that the ligand remains stable and available for binding to its receptor, even in the complex biological environment. The PEGylation provided by DSPE-PEG helps reduce non-specific binding to proteins and other molecules in the bloodstream, thus prolonging the circulation time of the liposomes and improving their bioavailability. The targeting ligands also facilitate specific cellular internalization via receptor-mediated endocytosis, allowing for more efficient and controlled delivery of peptide P33 directly into the target cells.

For example, when these liposomes were tested in vitro with cells overexpressing the folate receptor, fluorescence microscopy confirmed that the folic acid-functionalized liposomes exhibited increased uptake compared to non-functionalized liposomes, demonstrating the targeting specificity. In vivo, tumor-bearing animal models treated with RGD-functionalized liposomes showed enhanced accumulation of the liposomes at the tumor site, accompanied by greater therapeutic efficacy of the encapsulated peptide P33 compared to non-targeted liposomes.

In an embodiment, a dual-loading strategy is employed, wherein peptide P33 is encapsulated both in the liposomal core (aqueous phase) and within the lipid bilayer, with the latter being achieved by hydrophobic modification of peptide P33 via palmitoylation or myristoylation, and wherein the liposomal formulation is stabilized using a layer-by-layer (LbL) polyelectrolyte coating, wherein alternating layers of chitosan and hyaluronic acid are electrostatically deposited onto the vesicle surface, providing structural reinforcement and controlled release properties.

In this embodiment, a dual-loading strategy is employed to encapsulate peptide P33 in both the liposomal core (aqueous phase) and within the lipid bilayer, offering a unique and enhanced method of peptide delivery. This dual encapsulation allows for a two-pronged release mechanism, where the peptide can be gradually released from both the core and the bilayer, potentially enhancing the overall therapeutic effect by providing sustained release over time. The peptide encapsulation in the liposomal core involves traditional encapsulation techniques, where the peptide is dissolved in the aqueous phase during liposome formation. However, to achieve the loading within the lipid bilayer, the peptide is subjected to hydrophobic modification, such as palmitoylation or myristoylation, which attaches a fatty acid chain to the peptide. This modification makes the peptide more hydrophobic, enabling it to integrate into the hydrophobic region of the lipid bilayer.

By incorporating peptide P33 within the lipid bilayer, the release profile of the peptide becomes more controlled and gradual, as the peptide is shielded within the liposomal structure until external stimuli (such as pH changes or membrane disruption) occur. This strategy allows for the dual-controlled release of peptide P33 from two distinct locations within the liposomal formulation, which may be beneficial in maintaining therapeutic levels of the peptide over a prolonged period and enhancing its bioavailability in target tissues. The lipid bilayer-associated peptide also has the potential to provide a more localized release upon reaching the target site, particularly in situations where the liposome membrane is sensitive to stimuli (such as acidic pH in tumor environments), thus enhancing the specificity and efficacy of the treatment.

To further stabilize the liposomal formulation, a layer-by-layer (LbL) polyelectrolyte coating is applied, which involves the alternating deposition of positively charged and negatively charged polyelectrolytes onto the surface of the liposomes. In this embodiment, chitosan (a positively charged polysaccharide) and hyaluronic acid (a negatively charged glycosaminoglycan) are used as the alternating layers. The LbL technique takes advantage of electrostatic interactions between the charged molecules, which leads to the controlled deposition of each layer onto the liposomal surface. The result is a multilayered coating that provides enhanced structural reinforcement to the liposomes, making them more resistant to mechanical stress, membrane instability, and premature peptide release. This coating also plays a significant role in the control of peptide P33 release: the multilayer coating can act as a barrier to slow the diffusion of the peptide from both the core and the bilayer, allowing for a more sustained and regulated release over time.

The LbL coating also provides the potential for targeting and functionalization, as the hyaluronic acid and chitosan layers can be further modified with targeting ligands, such as folic acid or RGD peptides, for enhanced receptor-mediated delivery to specific cells or tissues. The hyaluronic acid layer, in particular, can bind to CD44 receptors overexpressed on certain cancer cells, promoting targeted delivery to those cells. In addition, the LbL coating contributes to the biocompatibility of the liposomes, as both chitosan and hyaluronic acid are natural polymers known for their low toxicity and immune-evading properties. This makes the formulation more suitable for in vivo applications, where minimizing immune response and promoting longer circulation times are crucial for achieving effective therapeutic outcomes.

For example, in vivo studies have shown that liposomes with a dual-loading strategy and LbL coating exhibit improved stability, as indicated by reduced aggregation and size changes during circulation in the bloodstream. These liposomes also demonstrate a sustained release profile, where the peptide is gradually released from both the core and the lipid bilayer, contributing to prolonged therapeutic effects. The LbL coating significantly contributes to this sustained release by acting as a barrier to rapid release, while still allowing for eventual release through hydrolytic degradation or external stimuli (such as pH or enzymatic activity) at the target site.

This dual-loading strategy, coupled with the LbL polyelectrolyte coating, represents an innovative approach to peptide delivery. It enables enhanced retention of peptide P33 within the liposomal formulation, ensuring a controlled and sustained release profile. The additional benefits of targeted delivery and structural reinforcement provided by the LbL coating make this embodiment particularly effective for targeted therapies where long-lasting and specific peptide release is needed. This approach also significantly improves the stability, efficacy, and bioavailability of the peptide-based formulation, offering a non-obvious and novel strategy for drug delivery systems that can be fine-tuned to meet the needs of various therapeutic applications, such as cancer treatment or other diseases requiring targeted peptide delivery.

In an embodiment, the peptide P33-loaded liposomes are embedded into a self-assembling peptide hydrogel network, wherein the gel forms a depot system that modulates peptide P33 diffusion kinetics upon administration, and wherein a gas vesicle protein shell derived from cyanobacteria is incorporated into the lipid bilayer, enabling ultrasound-triggered cavitation for controlled disruption and targeted peptide P33 delivery.

In this embodiment, peptide P33-loaded liposomes are incorporated into a self-assembling peptide hydrogel network, creating a sophisticated depot system that significantly enhances the control over peptide release kinetics following administration. The self-assembling peptide hydrogel is a type of bio-responsive material that, upon contact with physiological conditions (such as body temperature or pH), spontaneously forms a gel structure. This gel network provides a scaffold that encapsulates the peptide-loaded liposomes, allowing for the gradual release of the encapsulated peptide P33 over an extended period. The hydrogel's properties can be tailored by adjusting the peptide composition, concentration, and environmental conditions, providing a tunable diffusion system for peptide P33. As a result, the hydrogel system ensures a sustained release of peptide P33, enhancing its therapeutic efficacy by maintaining optimal drug concentrations over time. This approach is particularly advantageous for local and sustained drug delivery, such as in the treatment of chronic diseases or tumors, where maintaining steady therapeutic levels of the drug is critical.

The use of this peptide hydrogel network also contributes to the biocompatibility and biodegradability of the delivery system, as peptide-based hydrogels are inherently less toxic than synthetic polymers. The hydrogel also protects the encapsulated peptide P33 from premature degradation in vivo, further enhancing its stability and efficacy. The self-assembling nature of the hydrogel reduces the need for complicated fabrication processes and allows for the formulation to be easily adapted to specific delivery routes, such as intratumoral, subcutaneous, or intravenous injections.

Additionally, to further enhance the control over peptide P33 release and improve targeting capabilities, a gas vesicle protein shell derived from cyanobacteria is incorporated into the lipid bilayer of the liposomes. Gas vesicles are naturally occurring, proteinaceous structures found in certain microorganisms, such as cyanobacteria, that are capable of encapsulating gas and imparting buoyancy to the organism. In this embodiment, the gas vesicle protein shell serves a dual purpose: it acts as a structural component of the liposomal bilayer, enhancing the stability and mechanical properties of the liposome, and it also provides a novel trigger mechanism for controlled drug release via ultrasound-triggered cavitation.

Upon exposure to an ultrasound field, the gas vesicles undergo cavitation—a process in which small gas bubbles rapidly expand and collapse due to changes in pressure caused by the ultrasound waves. This cavitation effect leads to the disruption of the liposomal membrane, causing the controlled release of the encapsulated peptide P33. The presence of the gas vesicle shell makes the liposomes more responsive to ultrasound energy, allowing for targeted delivery of the peptide to specific tissues or organs, which can be identified using ultrasound imaging. This non-invasive method allows for precise spatial control over where and when the peptide is released, providing an effective strategy for localized drug delivery in tissues such as tumors, where ultrasound can be focused on specific areas.

For example, in in vivo applications, the liposomes can be injected into the bloodstream, where they circulate and are taken up by tissues or tumors. When the target tissue is reached, an external ultrasound transducer can be used to focus ultrasound waves on the region of interest. The gas vesicle-loaded liposomes, upon exposure to the ultrasound field, will undergo cavitation, leading to the rupture of the liposomal membrane and the controlled release of peptide P33 at the target site. This technique has been shown to be particularly effective in tumor-targeted therapies, where the combination of ultrasound-mediated cavitation and targeted drug release can lead to increased therapeutic efficacy while minimizing systemic side effects.

Moreover, the gas vesicle incorporation also adds a diagnostic feature to the system, as the gas-filled vesicles can be detected by ultrasound imaging. This capability enables real-time monitoring of the location of the liposomes within the body, offering a valuable tool for image-guided therapy. The ability to monitor the distribution of the liposomes and confirm their accumulation at the target site allows for more precise treatment and adjustment of therapeutic protocols in response to dynamic changes in the patient's condition.

### Materials

For the in vitro analysis, phospholipid including granulated soy lecithin, obtained from Lipoid GmbH (Germany), is a refined and granulated soy lecithin that contains 90% phosphatidylcholine. Cholesterol (powder form) is purchased from Sigma-Aldrich (Germany). Chloroform and methanol are purchased from Sigma-Aldrich (Germany). HEPES buffer is obtained from Sigma-Aldrich (Germany). Enterococcus faecalis ATCC 29212 is obtained from ATCC, USA.

For the computational analysis, the 3D structure of the receptor 50S ribosomal protein L16, present in Enterococcus faecalis (strain ATCC 700802), is obtain from the AlphaFlod server in PDB format.

### Methods

### Computational Analysis

### Molecular Docking Analysis and MD Simulation of the Receptor-Peptide Complex

The HPEPDOCK server is used to dock the PDB format for the ribosomal protein L16 to the amino acid sequence of the AMP P33. The receptor Q839F7 is also docked to the amino acids sequence of the antibiotic peptide Gramicidin using the HPEPDOCK server, to better understand the inhibitory effect of the peptide P33 in comparison to the antibiotic peptide Gramicidin. The GalaxyPepDock server is used to generate the PDB format for the receptor-peptide complexes Q839F7-P33 and Q839F7-Gramicidin. The resulting receptor-peptide complexes are then submitted to the Protein-Ligand Interaction Profiler (PLIP) website to analyze the results of the receptor-peptide docking interactions. Discovery Studio is used to visualize the binding site interactions. The PRODIGY server is utilized to explore the binding energy of the receptor-peptide interactions.

The interactions and stability of the docked Q839F7-P33 complex are evaluated using MD simulation. The complete MD simulation process is conducted on the Maestro Desmond Module. Prior to initiating the MD simulation, the OPLS3e force field is used to minimize the system's energy. We employed the TIP3P solvent model, and the addition of 23 Cl⁻ ions neutralized the system. The system is simulated for 50.098 ns at 300 K temperature and 1.01 bar of pressure (bar).

### In Vitro Analysis

### Preparation of Peptide P33

Synthesis of Peptide P33 with >95% purity is carried out by GenScript Biotech Corporation. The peptide P33 is dissolved in DMSO and diluted in water to obtain the final concentration of 200 μg/mL (DMSO <10%).

### Preparation of Liposome-Encapsulated P33

Liposomes are prepared using the conventional lipid film hydration method with some modifications. A lipid mixture of phospholipid including granulated soy lecithin (80 mg) and cholesterol (20 mg) is dissolved in 10 mL chloroform/methanol solvent with a molar ratio of 2:1. The organic solvent is evaporated using a magnetic stirrer (with the coupled heating system) until the lipid film is fully hydrated. Solvent traces are removed by drying in a vacuum overnight. The lipid film is then rehydrated with 10 mL HEPES buffer (pH 7.2) containing the peptide P33 (200 μg/mL). A magnetic stirrer (without heating) is used to thoroughly mix the solution until a thin lipid film is formed. The resulting lipid suspension is sonicated until a clear, translucent solution is obtained and extruded using a vortex to form large unilamellar vesicles (LUVs) with a uniform size of approximately 200 nm.

### Determination of Encapsulation Efficiency

The encapsulation efficiency of the peptide P33 in the prepared liposome formulation is determined by a method described earlier. Approximately 10 mL of liposome formulation is centrifuged at 15000×g for 30 minutes at 4° C. After centrifugation, the supernatant is extracted with a micropipette, diluted with methanol, and examined for the peptide P33 content using a UV-visible spectrophotometer (UV-1280) at a maximum wavelength of 300 nm. Another 10 mL sample of the liposome formulation is examined for the peptide P33 using UV absorbance (without centrifugation). The protein content within the prepared liposome formulation is estimated by monitoring their A₂₈₀ and A₂₆₀. The encapsulation efficiency % of the peptide P33 in the liposome formulation is calculated from the following equation,

$$Encapsulation efficiency \% = \frac{Amount of encapsulated P 33}{Amount of total P 33} \times 100$$

### Bactericidal Activity Assay of Liposome-Encapsulated P33

The broth dilution method is used to estimate the minimum inhibitory concentration (MIC) and minimum bactericidal concentration (MBC) to assess the bactericidal efficacy of the liposome preparation against E. faecalis ATCC 29212. To examine the improvement in the antibacterial activity after encapsulation, the MIC and MBC of the free peptide P33 are evaluated against the E. faecalis ATCC 29212. Briefly, bacterial colonies (1.5×10⁸ CFU/mL) are loaded into the wells of a polystyrene 96-well plate containing two-fold concentrations of the liposome preparation. The same bacterial colonies (1.5×10⁸ CFU/mL) are loaded into the wells of another polystyrene 96-well plate containing two-fold concentrations of the free peptide P33 and incubated at 35° C. for 24-36 hours. The MIC is defined as the lowest concentration of liposome preparation (or the free peptide) that produced a noticeable suppression of bacterial growth. The MBC is evaluated by subculture of samples from each well (10 μL) on Mueller Hinton Agar (MHA) medium and incubating at 35° C. for 24-36 hours. The MBC is defined as the concentration yielding an absence of bacterial growth (no colonies forming units (CFUs) are observed).

### Physicochemical Characterization of Liposomes

Dynamic light scattering is used to evaluate the mean particle diameter, polydispersity index (PDI), and zeta potential (electrophoretic mobility) of liposomes. The particle sizes for 1 mL samples are measured for a duration of 60 s at temperature 25° C. with a measurement position of 0.85 mm. The attenuator index 6 is considered optimal for the measurements. For the zeta potential measurement, up to 80 runs are performed.

### Statistical Analysis

Assays are conducted in triplicate, and values are reported as mean±standard deviation. Statistical significance is determined using the student t-test with a threshold of P=0.05.

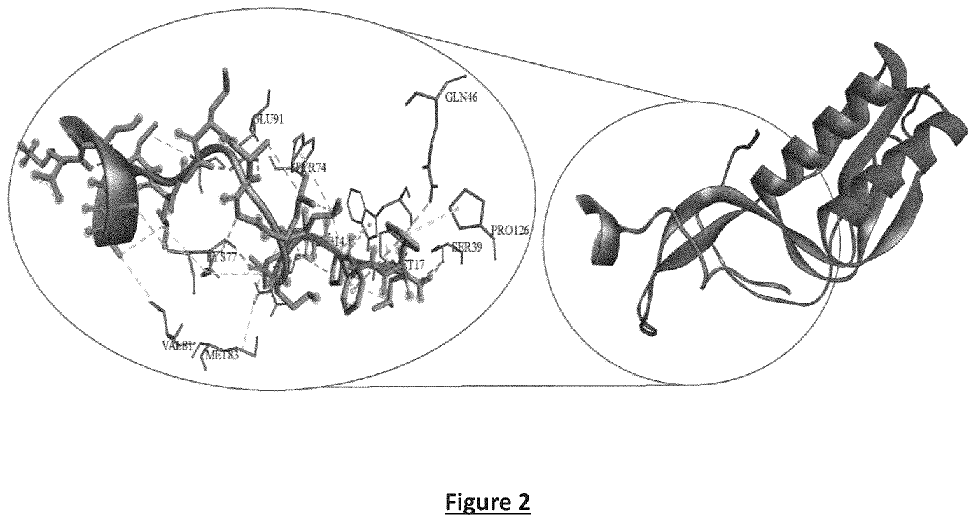

FIG. 2 illustrates a peptide P33 and protein Q839F7 binding site interactions visualized by Discovery Studio, 3D structure is generated by GalaxyPepDock server.

### Computational Analysis

### Molecular Docking Analysis and MD Simulation of the Receptor-Peptide Complex

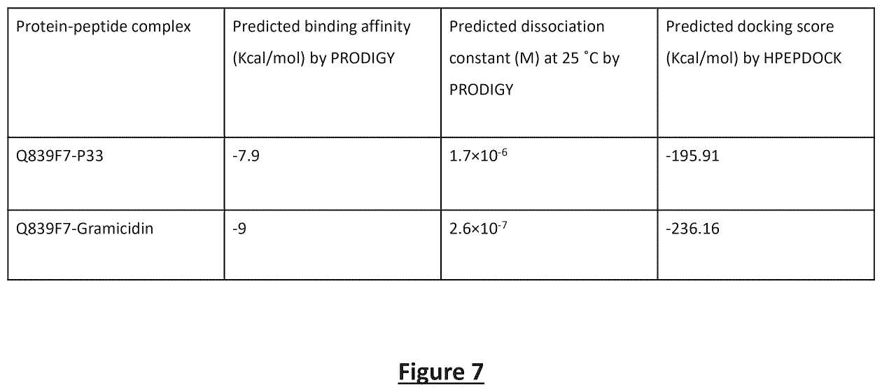

The structure of the Enterococcus faecalis ribosomal protein L16 (UNIPROT code: Q839F7) is docked to the antimicrobial peptide P33 and compared to the docking of the same receptor, the protein Q839F7, with the antibiotic peptide Gramicidin. The docking score of the receptor-peptide complex Q839F7-P33 predicted by HPEPDOCK server (TABLE in FIG. 7.) is −195.91 Kcal/mol, whereas the binding affinity predicted by PRODIGY for the same complex is −7.9 Kcal/mol. Gramicidin showed a more stable and energetically favorable binding mode with the protein Q839F7 when analyzed by PRODIGY (−9 Kcal/mol) and HPEPDOCK (−236.16 Kcal/mol), nevertheless, the difference in affinity between the peptide P33 and the antibiotic peptide Gramicidin is relatively small, suggesting a promising antibiotic activity for the peptide P33.

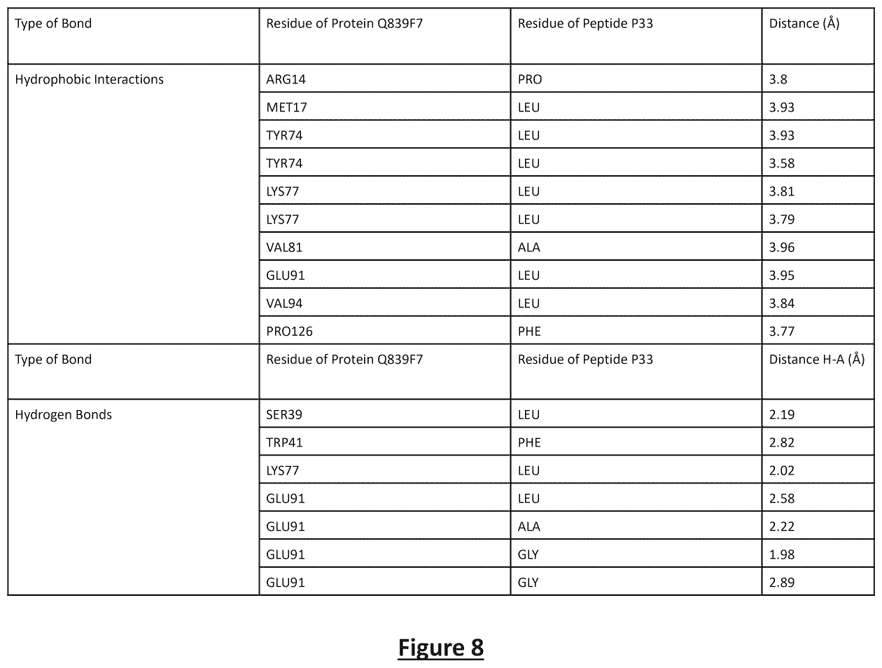

Interestingly, the analysis of the protein-peptide complex Q839F7-P33 interactions by PLIP server (FIG. 2, and TABLE in FIG. 8.) showed that the peptide P33 formed 4 hydrogen bonds, less than 3 Å (H-A) in length, with the active site residue Glu91.

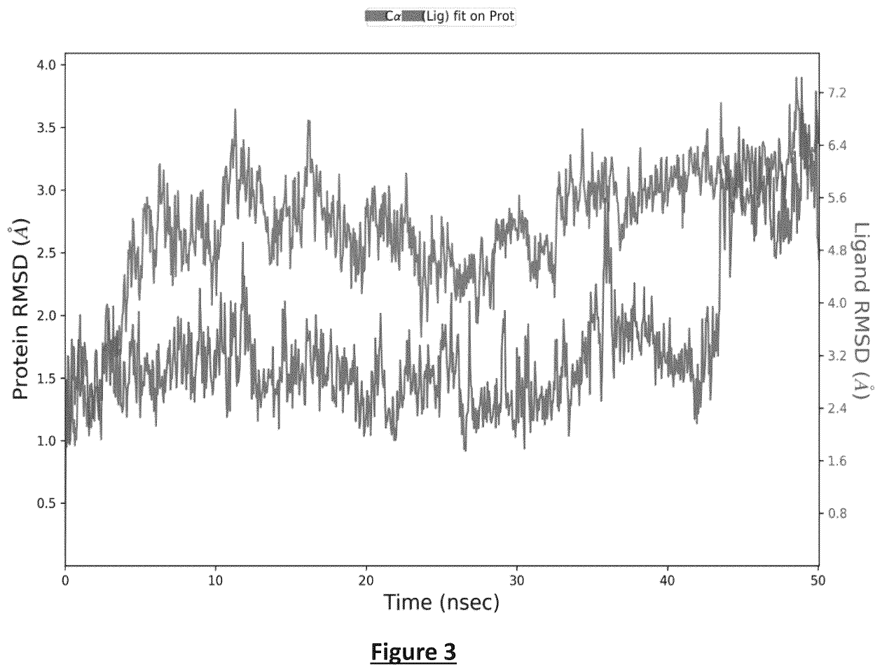

FIG. 3 illustrates an interaction between the protein Q839F7 and the peptide P33 during the 50 ns simulation. The RMSD plot displays P33 and protein C-alpha atoms. During 50 ns of simulation, indicating the stability of the bound peptide P33 in the binding pockets of the protein. In the RMSD plot of the Q839F7-P33 complex, the peptide P33 aligned with the protein Q839F7 backbones from the beginning of the simulation, followed by a small fluctuation, then aligned again until the end of simulation time.

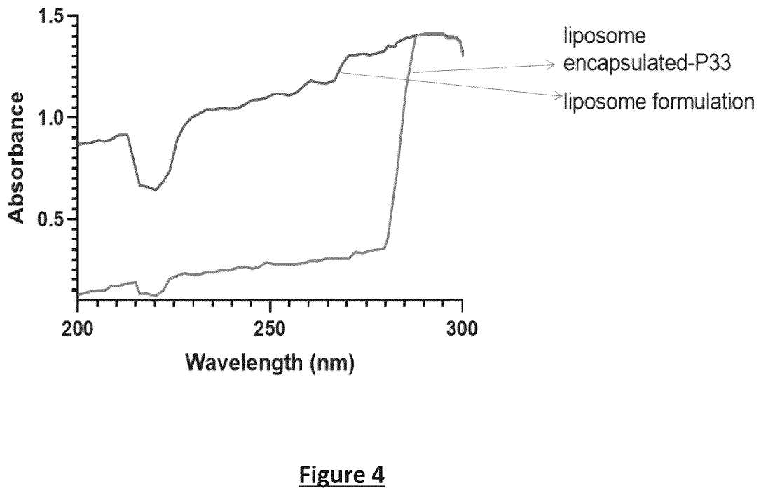

FIG. 4 illustrates absorbance spectra of liposome formulation containing both encapsulated and unencapsulated P33, compared to the absorbance spectra of liposome encapsulated-P33.

### In Vitro Analysis

### Determination of Encapsulation Efficiency

The encapsulation efficiency, defined as the percentage of the peptide that is successfully incorporated into the liposomes compared to the total amount used, is influenced by several factors, including the peptide's hydrophobicity, the liposome composition, and the encapsulation method employed. The encapsulation efficiency of the peptide P33 in the liposome formulation as estimated from UV absorbance (FIG. 4) is moderately high; 33.75%, suggesting optimization of liposome formulation. The relatively low encapsulation efficiency of the peptide P33 may be attributed to the hydrophobic nature of the peptide P33, since hydrophobic peptides tend to have low solubility in aqueous environments, which complicates their incorporation into liposome formulations.

### Bactericidal Activity Assay of Liposome-Encapsulated P33

For the MIC assay, the liposome formulation is prepared at the concentration of 1197 μg/mL and tested against E. faecalis ATCC 29212. The free peptide P33 is tested against the same strain E. faecalis ATCC 29212 and is prepared at the concentration of 200 μg/mL.

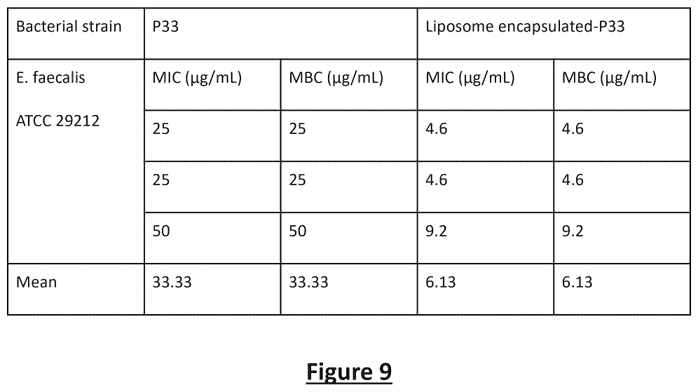

Results of the bactericidal activity assay (TABLE in FIG. 9.) showed that the free peptide P33 had an average MIC and MBC of 33.33 μg/mL, while the liposome-encapsulated P33 had a much lower average MIC and MBC of 6.13 μg/mL. This indicates that the liposome-encapsulated P33 is significantly more effective (P=0.032) against E. faecalis.

When compared to the MIC of common antibiotics like ampicillin or vancomycin against E. faecalis, an MIC of 33.33 μg/mL for P33 is moderate and suggests that while the peptide shows activity against E. faecalis, it may not be as potent as some conventional antibiotics, particularly when compared to clinical standards; reports indicate that some isolates of E. faecalis exhibit intermediate resistance with MICs ranging from 8 to 16 μg/mL. However, for the liposome-encapsulated P33, an MIC of 6.13 μg/mL lies in the range ampicillin-resistant strains that exhibited MICs of 4 to 8 μg/mL. The drastic reduction in the MIC and MBC for the liposome-encapsulated P33 suggests that encapsulation may enhance the peptide's stability, bioavailability, and interaction with the bacterial cell membrane.

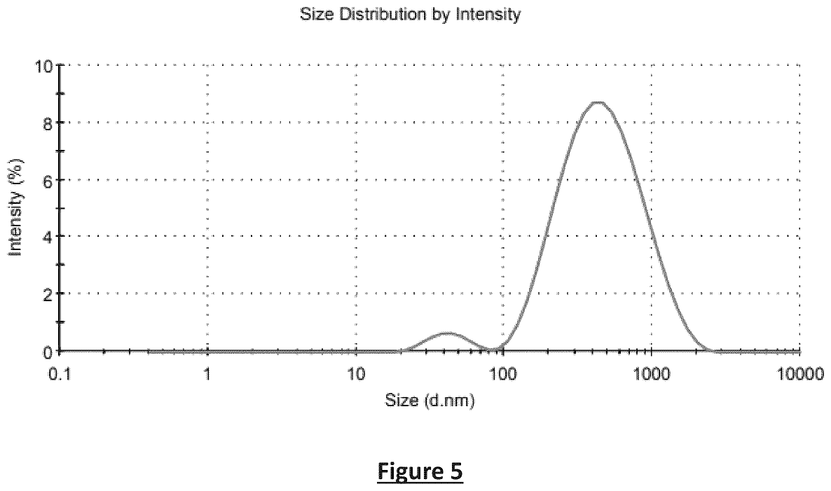

FIG. 5 illustrates size distribution intensity graph. Peak 1 had a size of 528.6 nm diameter and width of 333.9 nm diameter with an intensity of 96.4%. Peak 2 had a size of 43.04 nm diameter and width of 12.60 nm diameter with an intensity of 3.6%.

### Physicochemical Characterization of Liposomes

Results of particle size measurements are presented as size distribution by intensity (FIG. 5); the liposome formulation had an average particle diameter of 325.9 nm and a polydispersity index (PDI) of 0.423 which indicates a relatively uniform size distribution. While a PDI value of 0.423 is not extremely high, it does suggest that the liposome formulation should be optimized to reduce variability in size. A particle size in the range of 100-400 nm is often considered optimal for liposome formulations, as it facilitates enhanced cellular uptake and prolonged circulation time in the bloodstream

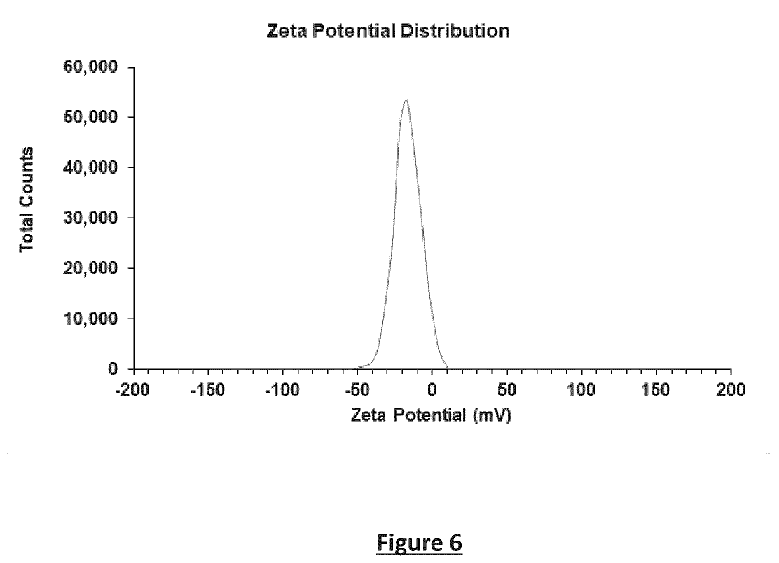

FIG. 6 illustrates an average total counts of 14445.13 the zeta potential is −16.95±23.19 mV. The liposome formulation is also negatively charged with a zeta potential of −16.95±23.19 mV (FIG. 6). Zeta potential is a critical parameter that reflects the surface charge of nanoparticles, influencing their stability in suspension. Generally, a zeta potential value below −30 mV or above +30 mV is indicative of good stability due to the electrostatic repulsion between similarly charged particles, which helps to prevent aggregation. In this case, the zeta potential of −16.95 mV suggests that while the liposomes may exhibit some stability, they are on the lower end of the spectrum, which could lead to potential aggregation over time.

FIG. 7 illustrates a table depicting molecular docking results of the antimicrobial peptide P33 and the antibiotic peptide Gramicidin with the protein Q839F7.

FIG. 8 illustrates a table depicting prediction of noncovalent interactions by PLIP server for the protein Q839F7 and the peptide P33 complex.

FIG. 9 illustrates a table depicting the MIC and MBC values (μg/mL) of the liposome encapsulated-P33 and the free peptide P33 against E. faecalis ATCC 29212.

The bactericidal efficacy of liposome-encapsulated antimicrobial peptide P33 is explored against Enterococcus faecalis, a major pathogen in dental infections, and compared to the bactericidal efficacy of the free peptide P33 against the same pathogen. The results of the in vitro analysis confirmed that liposome encapsulation significantly enhances the antimicrobial activity of P33. Supplemental computational analysis is performed to assess the inhibitory effect of the peptide P33 against the intracellular target 50S ribosomal protein L16, present in Enterococcus faecalis. Molecular docking and molecular dynamics simulation suggested promising interactions between P33 and the ribosomal protein L16 in E. faecalis, indicating a potential mechanism for its bactericidal action. The liposome-encapsulated P33 displayed considerably lower minimum inhibitory concentration (MIC) and minimum bactericidal concentration (MBC) values than the free peptide P33, suggesting increased antibacterial potency. Although the encapsulation efficiency is moderate (33.75%), the liposomes improved the peptide's stability and bioavailability, potentially increasing its effectiveness in clinical applications. The physicochemical characterization of the liposomes revealed suitable particle size and stability for potential therapeutic use. These findings suggest that liposome-encapsulated P33 holds promise as a more effective treatment for E. faecalis-related infections, with further optimization needed to improve encapsulation efficiency and formulation stability.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above about specific embodiments. However, the benefits, advantages, solutions to problems, and any component(s) that may cause any benefit, advantage, or solution to occur or become more pronounced are not to be construed as a critical, required, or essential feature or component of any or all the claims.

1. A process for preparing liposome-encapsulated peptide P33, comprising:
preparing a peptide P33;
preparing a lipid mixture by dissolving 70-90 mg of phospholipid including granulated soy lecithin and 20 mg of cholesterol in 15-25 mg of a chloroform/methanol solvent system;
evaporating organic solvent using a magnetic stirrer coupled with a heating system until a lipid film is fully formed and hydrated;
removing solvent traces by drying the lipid film under vacuum conditions overnight;
rehydrating the lipid film with 8-12 mL of a buffer containing 190-210 μg/mL of peptide P33;
mixing the rehydrated lipid film thoroughly using a magnetic stirrer at ambient temperature to ensure uniform dispersion;
sonicating the lipid suspension until a clear, translucent solution is obtained; and
extruding the resulting suspension using a vortex to form large unilamellar vesicles (LUVs) with a uniform size of approximately 200 nm.

2. The process of claim 1, wherein the lipid mixture comprises a molar ratio of phospholipids to cholesterol of 4:1 to optimize the encapsulation efficiency of peptide P33.

3. The process of claim 1, wherein the lipid film hydration process is modified by:
maintaining the buffer at a controlled pH of 7.2; and using a gradual mixing technique to enhance peptide encapsulation efficiency within the liposomes.

4. The process of claim 1, wherein the solvent comprises dimethyl sulfoxide (DMSO), wherein the DMSO concentration in the solution is less than 10%.

5. The process of claim 1, wherein the final concentration of the peptide in the solution is about 200 μg/mL.

6. The process of claim 1, wherein the molar ratio of phospholipid to cholesterol in the lipid film is about 2:1.

7. The process of claim 1, wherein the buffer is HEPES buffer.

8. The process of claim 1, wherein the weight amount of the phospholipid including granulated soy lecithin, Cholesterol, Peptide P33, and HEPES Buffer, is, 85 mg, 20 mg, 200 μg/mL, and 10 mL respectively.

9. The process of claim 1, wherein the lipid mixture is exposed to a controlled ozonolysis process prior to film formation, wherein ozone gas at a concentration of 0.5-1 ppm is introduced for 5-10 minutes to selectively modify unsaturated phospholipids, altering membrane rigidity, and wherein the buffer comprises a negatively charged dendrimer, selected from polyamidoamine (PAMAM) or polypropylene imine (PPI) at a concentration of 0.01-0.05 mg/mL, facilitating electrostatic interaction with peptide P33 to enhance loading within the liposomal bilayer.

10. The process of claim 1, wherein a microbubble-assisted dispersion technique is employed during hydration, wherein 5% (v/v) perfluorocarbon gas microbubbles are introduced into the buffer, forming transient cavitation effects that enhance lipid layer separation and peptide encapsulation, and wherein the solvent removal step is modified by applying a supercritical fluid-assisted drying technique, wherein carbon dioxide at 31.1° C. and 7.38 MPa is used to replace organic solvents without thermal degradation of lipids or peptide P33.

11. The process of claim 1, wherein a bioinspired cholesterol analog, selected from stigmasterol or lanosterol, is substituted for up to 30% of the cholesterol content in the lipid mixture, altering the fluidity and permeability characteristics of the liposomal bilayer, and wherein a phase-transition-controlled hydration method is employed, wherein lipid hydration occurs at a temperature above the gel-to-liquid crystalline phase transition temperature (Tm) of the phospholipids used, ensuring uniform lipid rearrangement and bilayer stability.

12. The process of claim 1, wherein the extrusion step is combined with an electroporation process, wherein an electric pulse of 0.5-1 kV/cm for 5-10 milliseconds is applied post-extrusion to induce transient nanopores in liposomal membranes, facilitating peptide P33 incorporation into the inner bilayer, and wherein the liposome suspension is subjected to acoustic levitation-assisted solvent exchange, wherein standing wave ultrasound fields (20-40 kHz) create a controlled evaporation environment to gradually remove residual organic solvent while preventing lipid aggregation.

13. The process of claim 1, wherein the final liposomal formulation is subjected to a hydrogel-embedded stabilization step, wherein the vesicles are suspended in a thermosensitive hydrogel matrix comprising poloxamer 407 or methylcellulose, preventing aggregation and prolonging peptide retention, and wherein the lipid film is hydrated using a gradient osmotic shock technique, wherein hydration is initiated with a hypotonic buffer followed by a gradual transition to an isotonic buffer to induce controlled vesicle swelling and enhance peptide P33 loading.

14. The process of claim 1, wherein the liposomal formulation is subjected to a reversible thermal annealing process, wherein the suspension is cycled between 4° C. and 42° C. for three cycles to induce lipid bilayer reorganization and reduce vesicle polydispersity, and wherein a photo-crosslinkable phospholipid, such as bis-SorbPC (1,2-bis(2-sorbyloxy-1,3-propadiyloxy) phosphatidylcholine), is incorporated into the lipid mixture and subjected to UV irradiation (365 nm) for 5-10 minutes, forming covalent crosslinks to improve vesicle stability.

15. The process of claim 1, wherein a stimuli-responsive lipid, selected from pH-sensitive dioleoylphosphatidylethanolamine (DOPE) or thermoresponsive dipalmitoylphosphatidylcholine (DPPC), is incorporated to enable controlled release of peptide, and wherein the peptide P33 is chemically conjugated to a hydrophobic anchor molecule, such as DSPE-PEG2000-maleimide, before liposomal encapsulation, allowing for improved retention within the lipid bilayer.

16. The process of claim 1, wherein the liposomal suspension is coated with a biomimetic lipid corona derived from decellularized exosome membranes, forming a hybrid lipid bilayer that enhances immune evasion and circulation stability, and wherein a charge-based lipid clustering strategy is used, wherein a cationic lipid, such as DOTAP (1,2-dioleoyl-3-trimethylammonium-propane), is introduced at 2-5 mol % to form electrostatic complexes with peptide P33, enhancing retention in the vesicle core.

17. The process of claim 1, wherein the final liposomal formulation is subjected to a high-frequency alternating magnetic field (AMF) treatment at 100-300 kHz in the presence of superparamagnetic iron oxide nanoparticles (SPIONs) embedded within the lipid bilayer, enabling triggered peptide P33 release upon external magnetic stimulation, and wherein the liposomal vesicles are functionalized with a targeting ligand, selected from transferrin, folic acid, or RGD peptides, via covalent conjugation to DSPE-PEG-maleimide to facilitate receptor-mediated uptake of peptide P33.

18. The process of claim 1, wherein a dual-loading strategy is employed, wherein peptide P33 is encapsulated both in the liposomal core (aqueous phase) and within the lipid bilayer, with the latter being achieved by hydrophobic modification of peptide P33 via palmitoylation or myristoylation, and wherein the liposomal formulation is stabilized using a layer-by-layer (LbL) polyelectrolyte coating, wherein alternating layers of chitosan and hyaluronic acid are electrostatically deposited onto the vesicle surface, providing structural reinforcement and controlled release properties.

19. The process of claim 1, wherein the peptide P33-loaded liposomes are embedded into a self-assembling peptide hydrogel network, wherein the gel forms a depot system that modulates peptide P33 diffusion kinetics upon administration, and wherein a gas vesicle protein shell derived from cyanobacteria is incorporated into the lipid bilayer, enabling ultrasound-triggered cavitation for controlled disruption and targeted peptide P33 delivery.
